(19) United States
(12) Patent Application Publication
(10) Pub. No.: US 2011/0071606 A1
(43) Pub. Date: Mar. 24, 2011

(54) BIFURCATED LEAD SYSTEM AND APPARATUS

(75) Inventors: John E. Kast, Hugo, MN (US); James A. Zimmerman, Blaine, MN (US); Craig S. Pilarski, Ham Lake, MN (US); William C. Phillips, Brooklyn Park, MN (US); Thomas I. Miller, Blaine, MN (US); Mary L. Boatwright, Andover, MN (US)

(73) Assignee: Medtronic, Inc., Minneapolis, MN (US)

(21) Appl. No.: 12/992,909

(22) PCT Filed: May 29, 2009
(86) PCT No.: PCT/US2009/045574

§ 371 Date: Nov. 16, 2010

Related U.S. Application Data
(60) Provisional application No. 61/058,284, filed on Jun. 3, 2008.
(60) Provisional application No. 61/175,476, filed on May 5, 2009.

(51) Int. Cl. A61N 1/05

(52) U.S. Cl. 607/116

(57) ABSTRACT

Bifurcated leads may simplify implantation procedures associated with electrical single therapy at two distinct anatomical locations, such as a left and a right occipital nerve.

### FIELD

The present disclosure relates to implantable medical devices; more particularly to medical leads capable of delivering electrical signals to two discrete anatomical locations, such as a left and a right occipital nerve.

### BACKGROUND

Headaches, such as migraines, and occipital neuralgia are often incapacitating and may lead to significant consumption of drugs to treat the symptoms. However, a rather large number of people are unresponsive to drug treatment, leaving them to wait out the episode or to resort to coping mechanisms. For refractive occipital neuralgia, nerve ablation or separation may effectively treat the pain.

Occipital nerve stimulation may serve as an alternative for treatment of migraines or occipital neuralgia. For example, a dual channel implantable electrical generator may be implanted subcutaneously in a patient. A distal portion of first and second leads may be implanted in proximity to a left and right occipital nerve such that one or more electrode of the leads are in electrical communication with the occipital nerves. The proximal portions of the leads may then be connected to the signal generator such that electrical signals can be delivered from the signal generator to the electrodes to apply therapeutic signals to the occipital nerves Alternatively, two single channel implantable electrical generators may be employed, where the first lead is connected to one signal generator and the second lead is connected to the second signal generator. In either case, the lead is typically tunneled subcutaneously from site of implantation of the signal generator to the occipital nerve or around the base of the skull. Such tunneling can be time consuming and is invasive.

### BRIEF SUMMARY

The present disclosure, among other things, describes leads, systems and methods for applying electrical signals to occipital nerves. In some embodiments, bifurcated leads are described. By using bifurcated leads, only one tunneling procedure is needed to tunnel a proximal portion of a lead between a location near the occipital nerves and the implantation site of the electrical signal generator. Such leads and procedures may reduce surgery time and invasiveness associated with occipital nerve stimulation.

In an embodiment, a method for applying electrical signals to a left occipital nerve and a right occipital nerve of a subject are described. The method includes providing a lead including (i) a proximal portion having first and second contacts and (ii) first and second distal arms. The first distal arm includes a first electrode, and the second distal arm includes a second electrode. The first electrode is electrically coupled to the first contact, and the second electrode is electrically coupled to the second contact. The method further includes placing the first electrode in electrical communication with the right occipital nerve, and placing the second electrode in electrical communication with the left occipital nerve. The method also includes generating a first electrical signal in an electrical signal generator implanted in the subject. The electrical signal generator is operably coupled to the lead via the first contact. The method additionally includes applying the first electrical signal to the right occipital nerve via the first electrode. The method also includes generating a second electrical signal in an electrical signal generator implanted in the subject. The electrical signal generator is operably coupled to the lead via the second contact. The method further includes applying the second electrical signal to the left occipital nerve via the second electrode of the lead. The first and second electrical signals are the same or different. It will be understood that a signal may be delivered between the first and second electrodes to apply the signal to the left or right occipital nerve in some circumstances.

In an embodiment, a bifurcated lead is described. The lead includes a proximal portion having first and second contacts, and includes a first distal arm having a first electrode electrically coupled to the first contact and having a first engagement element distal the electrode. The engagement element is configured to cooperate with an advancement tool such that advancement of the tool distally relative to the engagement element pushes the engagement element distally. The lead further includes a second distal arm having a second electrode electrically coupled to the second contact and having a second engagement element distal the electrode. The engagement element is configured to cooperate with an advancement tool such that advancement of the tool distally relative to the engagement element pushes the engagement element distally. The lead also includes a branch region where the lead transitions from the proximal portion to the first and second distal arms. In addition, the lead includes a tissue anchoring element attached to the branch region.

The leads, systems and methods described herein provide one or more advantages over prior leads, extensions, signal generators, systems and methods. Such advantages will be readily understood from the following detailed description when read in conjunction with the accompanying drawings.

### BRIEF DESCRIPTION OF THE DRAWINGS

FIG. 1 is a schematic side view of an implantable system including a signal generator, lead extension and lead.

FIGS. 2A-B are schematic diagrams showing distal portions of bifurcated leads implanted in a subjects and positioned to apply an electrical signal to left and right occipital nerves.

FIG. 3A is a schematic side view of a representative bifurcated lead.

FIGS. 3B-D are schematic cross-sections of alternative embodiments of the proximal portion of the lead shown in FIG. 3A taken through line 3b-3b.

FIG. 3E is a schematic side view of an embodiment of the branch region of the lead depicted in FIG. 3A, showing conductors running through the branch region.

FIG. 4 is a schematic side view of representative bifurcated leads.

FIGS. 5A-C are schematic drawings of lines running in a plane, showing embodiments of angles at which the receptacles of a connector portion of an extension may enter a body of the connector.

FIGS. 6-9 are schematic side views of representative bifurcated leads.

FIGS. 10A-E are schematic side views of representative bifurcated leads having extensible portions.

FIGS. 11A-F are schematic side views of representative bifurcated leads having attached anchors.

FIGS. 12A-B, 13, 14A-B, 15, and 16A-B are various views of schematic diagrams of embodiments of distal portions of leads having an engagement element.

FIGS. 17-19 are schematic side views of tools for engaging engagements elements, such as those depicted in FIGS. 12A-B, 13, 14A-B, 15, and 16A-B, to facilitate placement of a lead in a patient.

FIGS. 20A-C, 21A-B, 22A-D, and 23A-D are schematic views of engagement tools pushing leads via interaction with an engagement element.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

“Exemplary” or “representative” is used herein in the sense of “for example” or “for the purpose of illustration”, and not in a limiting sense.

The present disclosure describes, inter alia, bifurcated lead that may simplify implantation procedures associated with electrical single therapy at two distinct anatomical locations, such as a left and a right occipital nerve.

Nearly any implantable medical device or system employing leads may be used in conjunction with the leads, extensions or adaptors described herein. Representative examples of such implantable medical devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. For purposes of occipital nerve stimulation, electrical signal generators such as Medtronic, Inc.'s Restore® or Synergy® series of implantable neurostimulators may be employed.

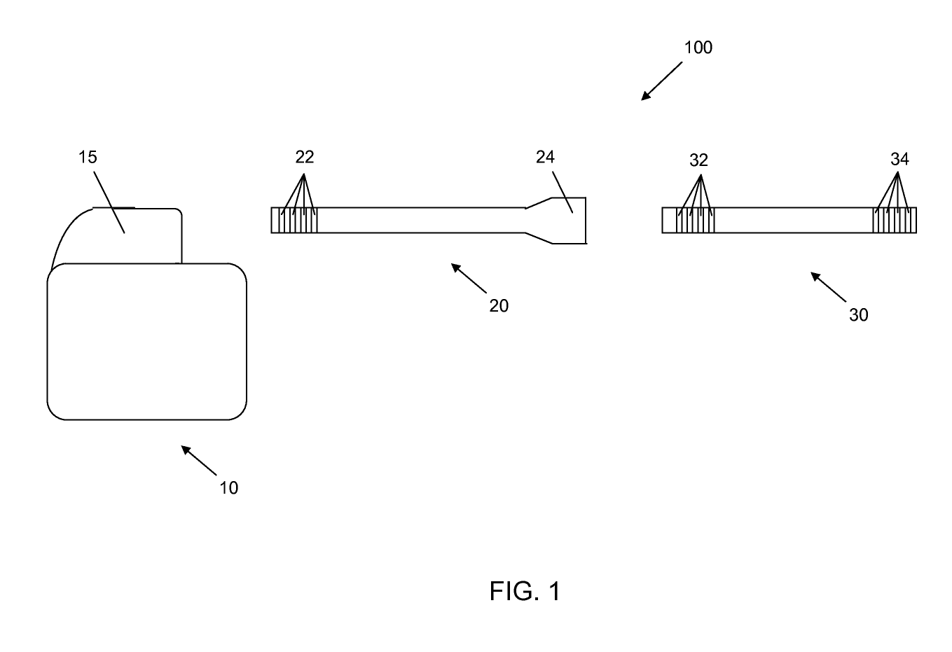

Referring to FIG. 1, a schematic side view of a representative electrical signal generator system 100 is shown. In the depicted system 100, the electrical signal generator 10 includes a connector header 15 configured to receive a proximal portion of lead extension 20. The proximal portion of lead extension 20 contains a plurality of electrical contacts 22 that are electrically coupled to internal contacts (not shown) at distal connector 24 of lead extension 20. The connector header 15 of the signal generator 10 contains internal contacts (not shown) and is configured to receive the proximal portion of the lead extension 20 such that the internal contacts of the connector header 15 may be electrically coupled to the contacts 22 of the lead extension 20 when the lead extension 20 in inserted into the header 15.

The system depicted in FIG. 1 further includes a lead 30. The depicted lead 30 has a proximal portion that includes a plurality of contacts 32 and a distal portion that includes a plurality of electrodes 34. Each of the electrodes 34 may be electrically coupled to a discrete contact 32. The distal connector 24 of the lead extension 20 is configured to receive the proximal portion of the lead 30 such that the contacts 32 of the lead 30 may be electrically coupled to the internal contacts of the connector 24 of the extension 20. Accordingly, a signal generated by the signal generator 10 may be transmitted to a patient by an electrode 34 of lead 30 when lead is connected to extension 20 and extension 20 is connected to signal generator 10.

It will be understood that lead 30 may be coupled to signal generator 10 without use of an extension 20. Any number of leads 30 or extensions 20 may be coupled to signal generator 10. Typically, one or two leads 30 or extensions 20 are coupled to signal generator 10. While lead 20 is depicted as having four electrodes 34, it will be understood that lead 30 may include any number of electrodes 34, e.g. one, two, three, four, five, six, seven, eight, sixteen, thirty-two, or sixty-four. Corresponding changes in the number of contacts 32 in lead 30, contacts 22 and internal contacts in connector 24 of lead extension, or internal contacts in connector 15 of signal generator 10 may be required or desired.

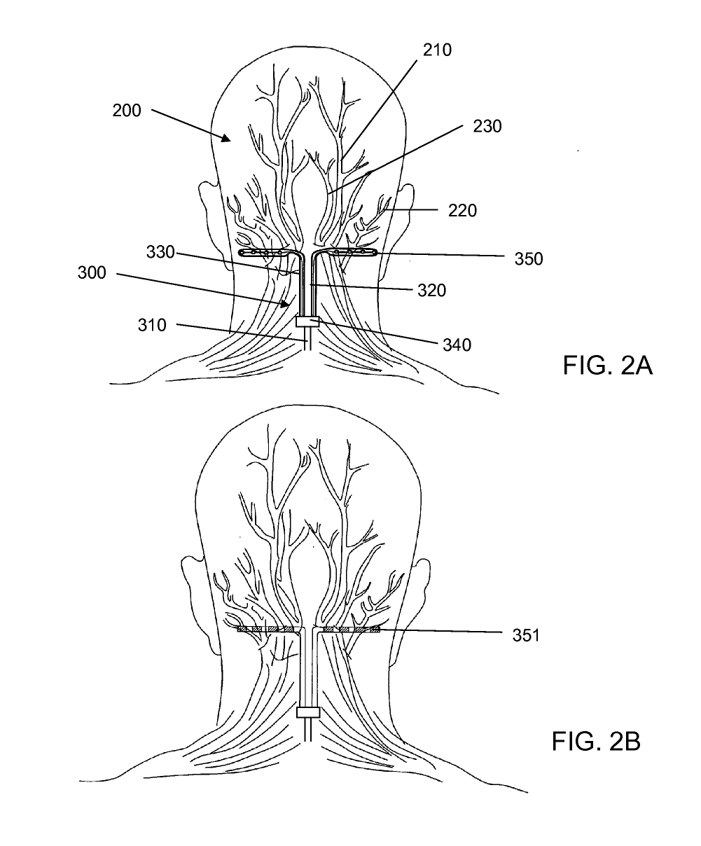

Referring to FIGS. 2A-B, bifurcated leads 300 are shown implanted in a patient to provide bilateral therapy to left and right occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions 350, 351 that contain electrodes are placed to allow bilateral application of electrical signals to the occipital nerve 200 at a level of about C1 to about C2 or at a level in proximity to the base of the skull. The position of the electrode(s) may vary. It will be understood that the electrode need not, and in various embodiments preferably does not, contact the nerve to apply the signal to the nerve. It will be further understood that a signal may be applied to any suitable portion of an occipital nerve, whether at a trunk, branch, or the like. In various embodiments, one or more electrodes are placed between about 1 cm and about 8 cm from the midline to effectively provide an electrical signal to the occipital nerve 200.

As shown in FIG. 2A, a bifurcated lead 300 may include a paddle shaped distal portion 350 containing electrodes. Such paddle shaped leads are often referred to as surgical leads. Examples of surgical leads that may be used or modified to form leads as described herein include Medtronic Inc.'s Resume, SymMix, On-Point, or Specify series of leads. Surgical leads typically contain electrodes that are exposed through one face of the paddle, providing directional stimulation. The depicted bifurcated lead 200 also includes a single proximal portion 310 that allows for only one tunneling procedure to the signal generator (not shown) implant site. In addition, the bifurcated lead 300 contains a branch region 340 and first 320 and second 330 distal arms. As shown in FIG. 2B, the bifurcated lead may include distal portion 350 that include electrodes that are generally cylindrically shaped. Such leads are often referred to percutaneous leads. Examples of percutaneous leads that may be used or modified to form leads as described herein include Medtronic Inc.'s Quad Plus, Pisces Quad, Pisces Quad Compact, or 1×8 SubCompact, 1×8 Compact, and 1×8 Standard leads. Such percutaneous leads typically contain ring electrodes that apply an electrical stimulation signal to tissue in all directions around the ring. Accordingly, the amplitude of the signal (and thus the energy required from the signal generator) applied may be greater with percutaneous leads that surgical leads for occipital nerve therapies.

Various embodiments of lead or system configurations are described below with reference to the figures discussed below. However, it will be understood that any bifurcated lead may be employed to apply an electrical signal to an occipital nerve; e.g., as described above with regard to FIGS. 2A-B. It will be further understood that, while the lead and system configurations described below may be useful for applying electrical signals to occipital nerves, they may be employed to apply electrical signals to other tissues of a subject or may be used to record signals from tissue of a subject.

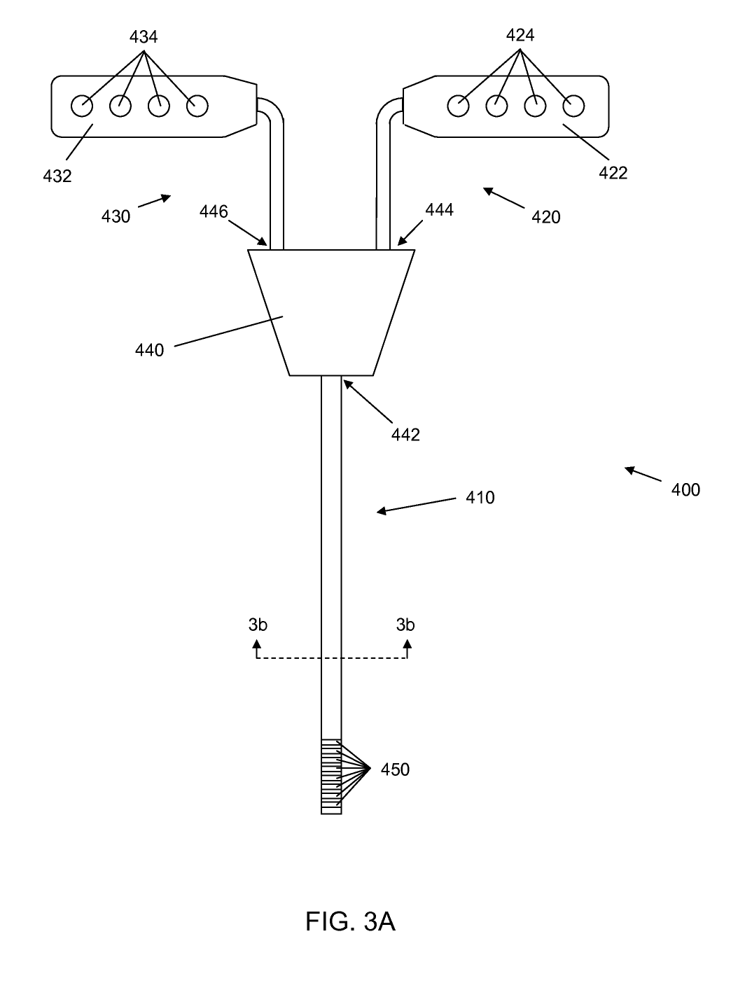

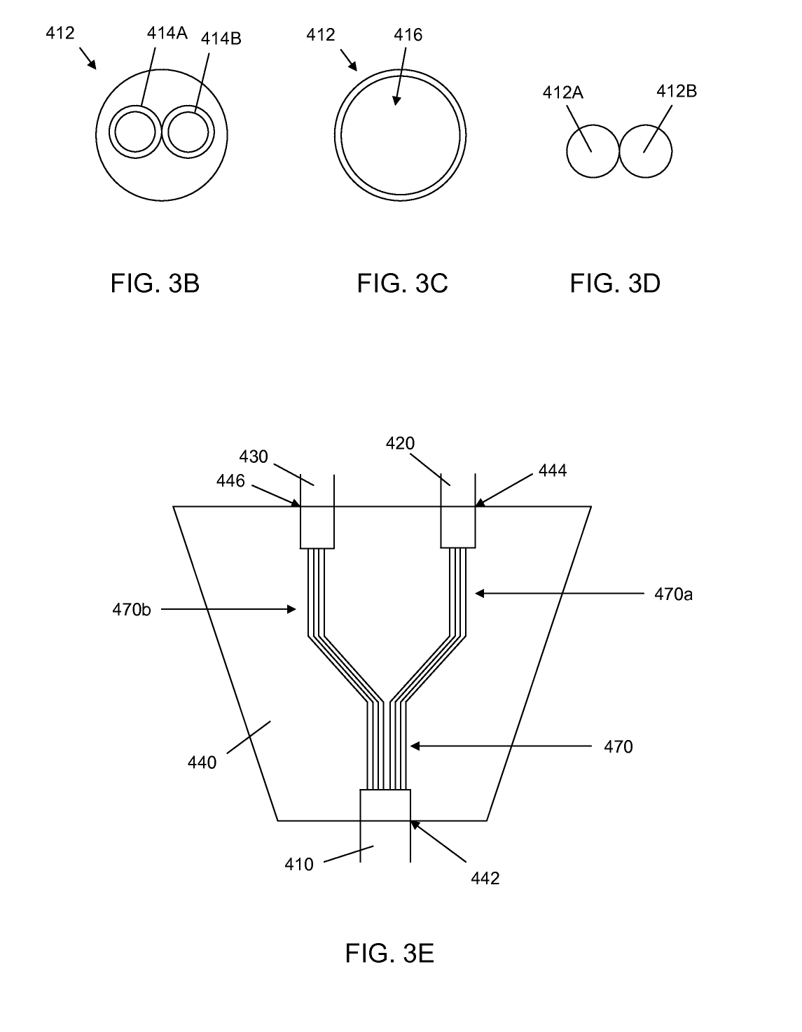

Referring now to FIG. 3A, a schematic side view of a representative bifurcated lead 400 is shown. The lead 400 includes a proximal portion 410 that includes a plurality of contacts 450 for electrically coupling to an electrical signal generator, lead extension, adaptor, or the like. The lead 400 also includes first 420 and second 430 distal arms that contain electrodes 424, 434. The electrodes 424, 434 are electrically coupled to contacts 450 via conductors that run within lead 400 from the contacts 450 to the electrodes 424, 434. The lead 400 further includes a branch region 440 where the lead 400 transitions from the proximal portion 410 to the distal arms 420, 430. The branch region 440 may be of any suitable size and shape. In various embodiments, the branch region 440 has a volume of less than about 10 cubic centimeters; e.g., less than about 5 cubic centimeters.

Referring now to FIG. 3B-D, which are schematic cross sectional views of embodiments of the proximal portion 410 of the lead 400 depicted in FIG. 3A taken along line 3b-3b. As shown in FIG. 3B, the proximal portion of the lead includes a lead body 412. The lead body 412 may include two lumens or tubes 414A, 414B (or any number of tubes or lumens, e.g. one for each conductor) through which or around which conductors (not shown) may run to connect proximal contacts with electrodes of the first and second distal arms. Of course, the lumens or tubes 414A, 414B may be solid and the conductors can run in separate tracks along the length of the proximal portion of the lead until connecting with the distal arms. Alternatively, as shown in FIG. 3C, the lead body 412 in the proximal portion may include a single lumen 416 or solid core (not shown) and the conductors (not shown) may run in a single track along the along the length of the proximal portion of the lead. Alternatively, as shown in FIG. 3D, the proximal portion of the lead may include two attached lead bodies 412A, 412B through which separate channels of conductors (not shown) run. Of course, the lead body of the proximal portion of lead body may be configured in any other suitable manner.

Referring now to FIG. 3E, a representative example of a branch region 440 is shown in which the branch region 440 is transparent for purposes of illustration. In the depicted embodiment, a set of conductors 470 exit a lead body from the proximal portion 410 of the lead. The set of conductors 470 are separated into subsets 470a, 470b that independently enter lead bodies of the first 420 and second 430 distal arms. Any suitable manner of forming branch region 440 and separating conductors 470 for entry of subsets 470a, 470b into distal arms 420, 430 may be employed. For example, a lead body containing conductors 470 in proximal portion 410 may be formed. Additional lead bodies containing conductor subsets 470a, 470b forming distal arms 420, 430 may be formed. The conductor subsets 470a, 470b may be appropriately electrically coupled to the set of conductors 470 and branch region 440 may be overmolded over conductors 470, 470a, 470b, resulting in branch region 440 as depicted. Of course, any other suitable process may be employed to form branch region 440 and appropriately electrically couple proximal portion 410 of the lead to the distal arms 420, 430.

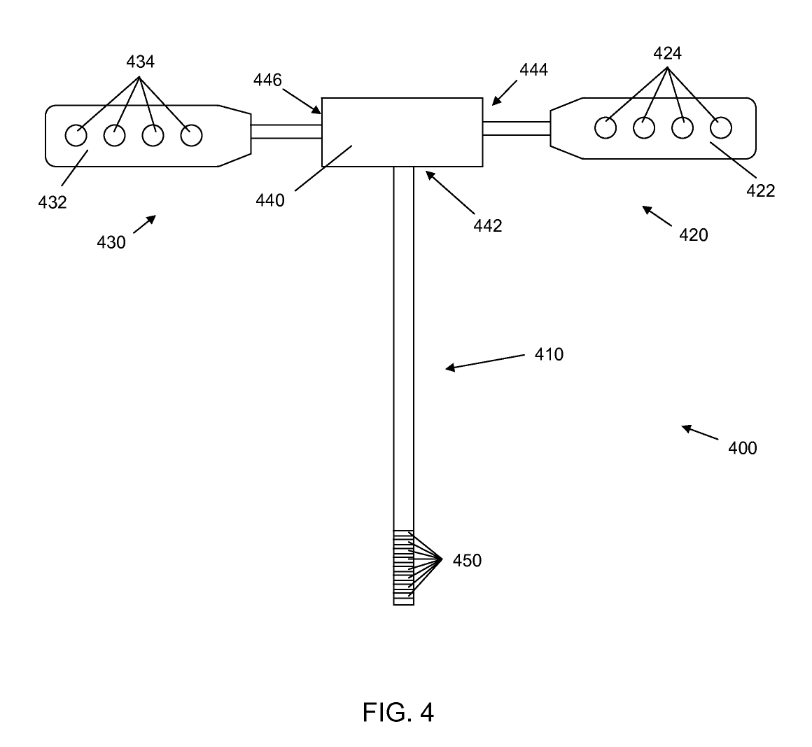

Referring now to FIG. 4, a schematic side view of a representative lead 400 is shown. The lead 400 includes a proximal portion 410 including contacts 450, a first distal arm 420 having a paddle-shaped region 422 containing electrodes 424, a second distal arm 430 having a paddle-shaped region 432 containing electrodes 434, and a branch point 440 where the lead 400 transitions from the proximal portion 410 to the first 420 and second 430 distal arms. The distal arms 420, 430 exit the branch region 440 at second 444 and third 446 entry regions, respectively. The proximal portion 410 enters the branched region 440 at the first entry region 442. The distal arms 420, 430 exit the branch point 440 substantially perpendicular to the angle of entry of the proximal portion 410 in the depicted embodiment. Of course, the distal arms 420, 430 may exit the branch region 440 at any suitable angle.

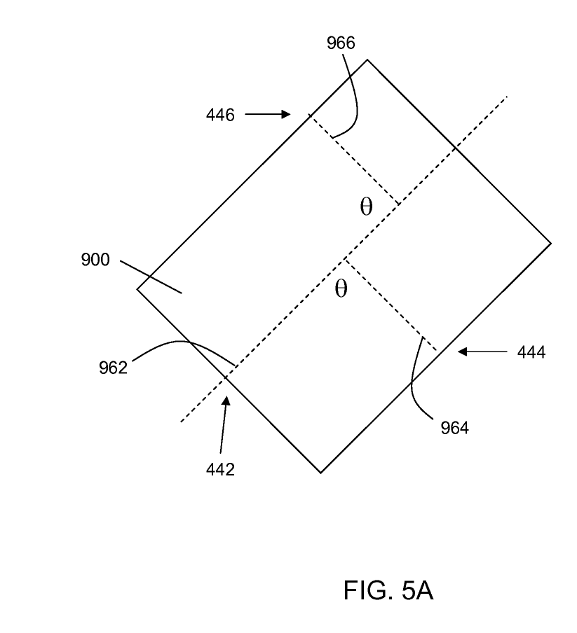

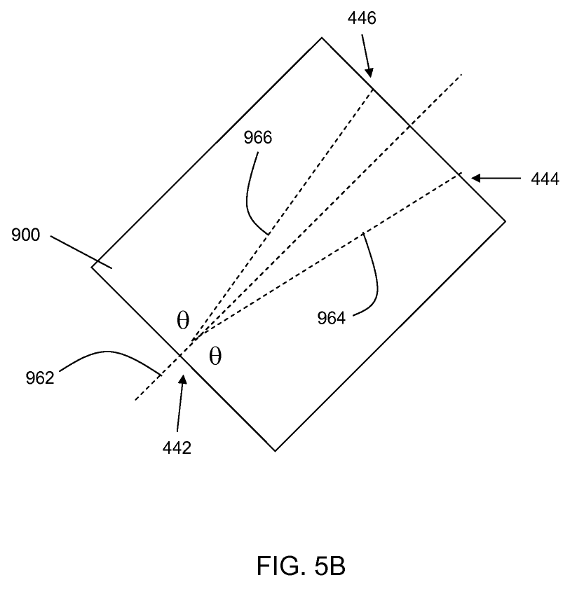

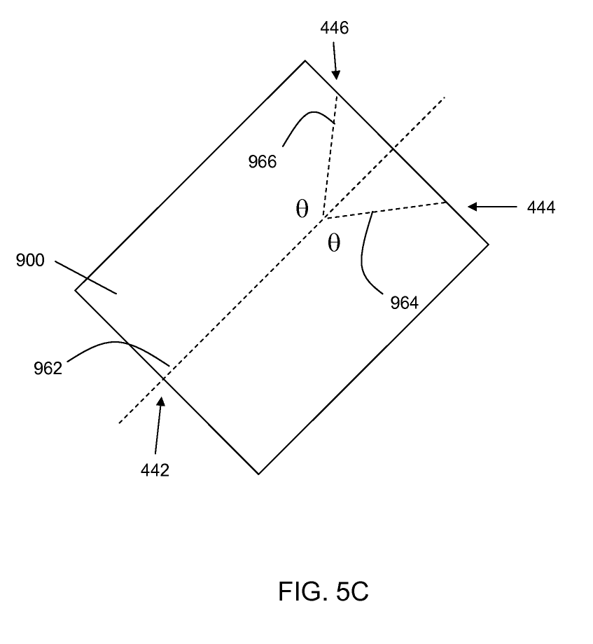

For example and with reference to FIGS. 5A-C, representative configurations are shown where the distal arms 420, 430 exit the branch region at various angles are shown. In FIGS. 5A-C, a plane 900 is shown. The plane 900 is defined by the geometric centers of the first entry region 442 where the proximal portion of the extension enters the connector, the second entry region 444 where the first distal arm exits the branch region, and the third entry region 446 where the second distal arm exits the branch region. Several lines 962, 964, 964 are shown running in the plane. Line 962 represents a line running through the geometric center of the entry point 442, along the axial center of the proximal portion of the extension as it enters the branch region. Line 964 represents a line running through the geometric center of the second entry point 444, along the axial center of the first distal arm as it exits the branched region. Line 966 represents a line running through the geometric center of third entry region 446, along the axial center of the second distal arm as it exits the branched region. In various embodiments, lines 962 and 964 or lines 962 and 966 intersect at angles (indicated by “A”) of between about 90 degrees and about 180 degrees. In some embodiments, the angles are between about 110 degrees and about 160 degrees.

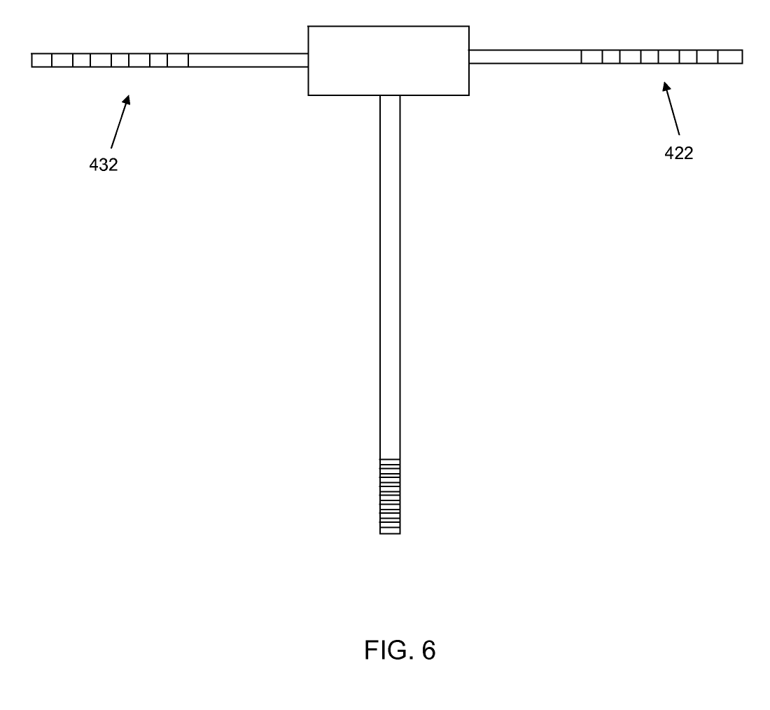

With reference to FIG. 6, an alternative configuration of an exemplary lead 400 is shown. In the embodiment depicted in FIG. 6, the distal portions 422, 432 containing the electrodes are substantially cylindrical (e.g., percutaneous-type). Of course, distal portions containing the electrodes may have any suitable shape.

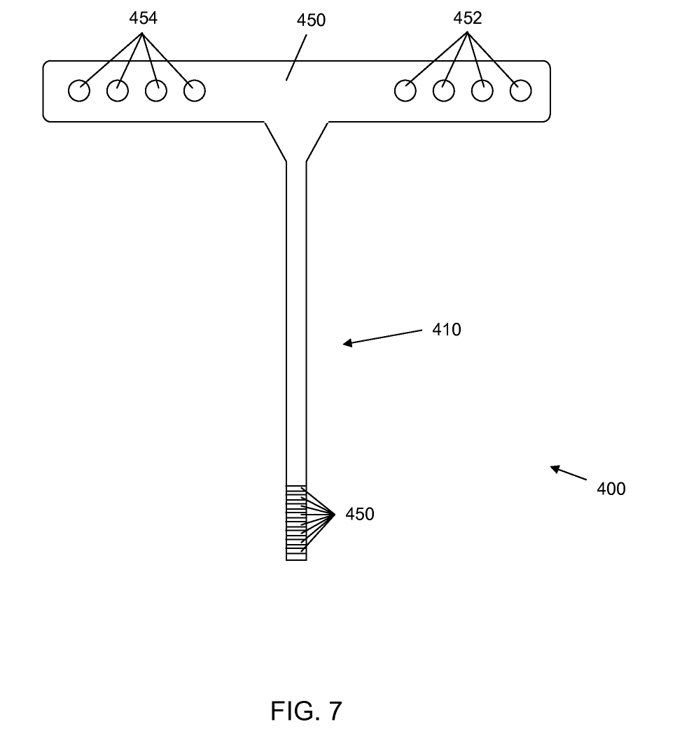

Referring now to FIG. 7, a schematic side view of a representative lead 400 is shown. The lead includes a proximal portion 410 containing contacts 450 and a distal portion 450 substantially perpendicular to the proximal portion 410. The distal portion 450 includes first 452 and second 454 sets of electrodes that are electrically coupled to the contacts 450. The first 452 and second 454 sets of electrodes are spaced apart from one another. In the embodiment depicted, the distal portion 450 can be considered to include two arms with one being to one side of the midline of the proximal portion 410 and the other being to the other side of the midline.

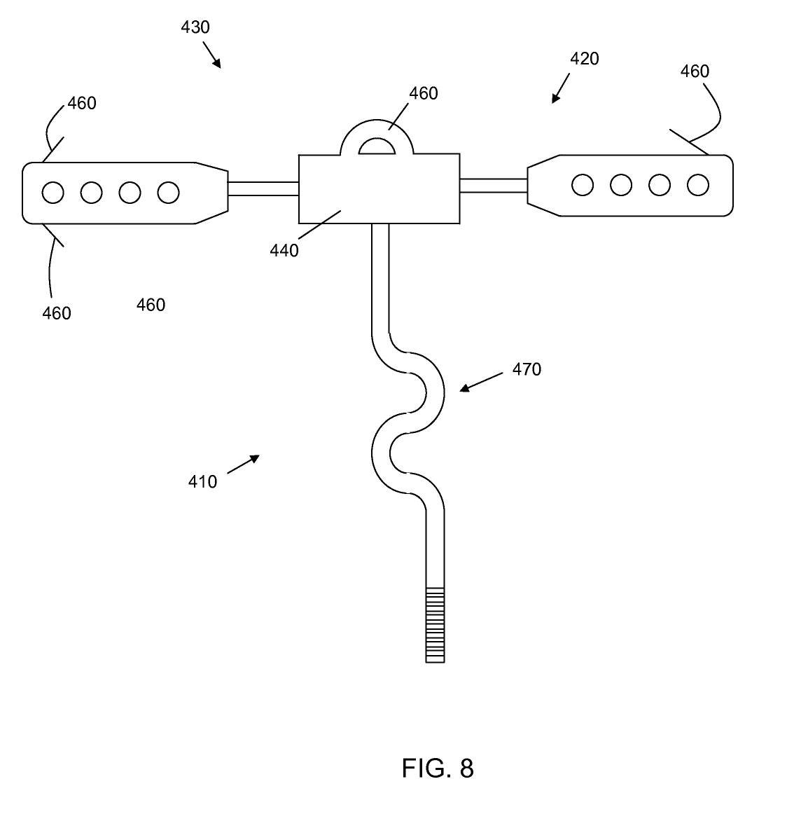

Referring now to FIG. 8, a lead 400 may include one or more anchors 460 for facilitating retention of the lead to tissue into which it is implanted. The anchors 460 may include suture holes or tines as depicted, but the anchors may take any suitable form. In various embodiments, an anchor 460 is attached to branch region 440. That is, the anchor 460 is secured in place on the branch region 460 prior to implantation. As used herein, “attached”, as it relates to an anchor and a branch region or the like, means the anchor is affixed to the branch region. The anchor is affixed well in advance of implantation; e.g., during manufacture of the lead. By way of example, the anchor may be fastened to, adhered to, integrally formed with, etc. the branch region. In various embodiments, the anchor is permanently attached to the branch region. For application of therapies to an occipital nerve, where proximal portion 410 is tunneled through the neck region of a subject, it may be desirable to securely anchor branch region 440 to tissue of the subject to prevent stress and strain placed on the proximal portion 410 of the lead from transferring to the distal arms 420, 430 through the branch region 440. In addition, it may be desirable for proximal portion to contain a strain relief feature to allow for stretching and movement of the neck (and thus proximal portion 410) without transferring excessive force to branch region 440. For example, proximal portion 410 may include a sigma shaped portion 470, may be looped (not shown), or may be extensible. One or more anchors 460 may be attached to first 420 or second 430 distal arms or to portions thereof, such as the distal portions containing electrodes as depicted.

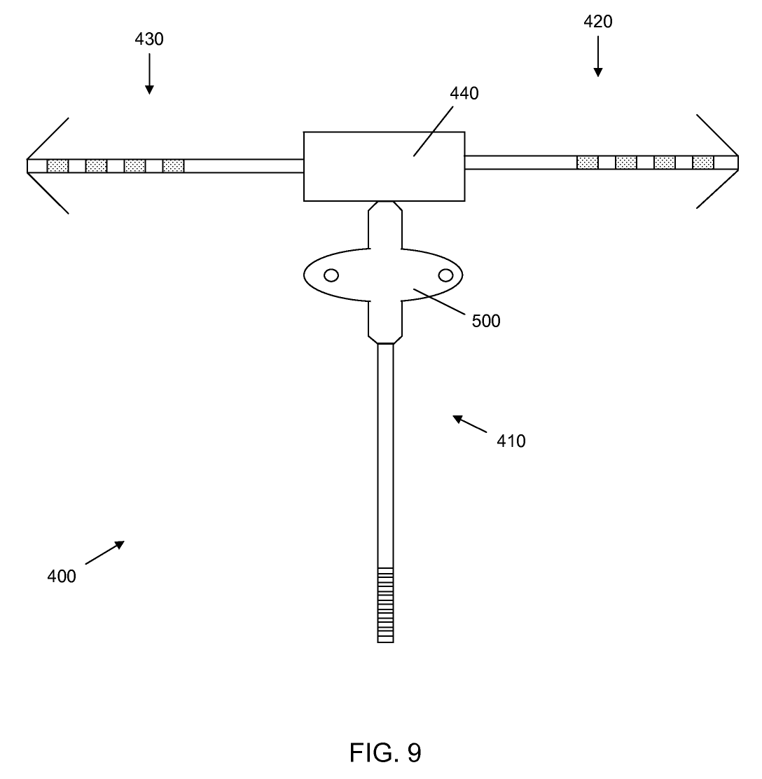

As depicted in FIG. 9, an unattached anchor 500, such as the wing-shaped suture loop anchor depicted, may be disposed about the proximal portion 410 of the lead 400 to prevent or inhibit strain on the lead 400 experienced proximal the anchor 500 from transferring to the branch region 440 and thus to the distal arms 420, 430. An unattached anchor 500 may be employed in addition to or alternatively to an attached anchor (e.g. as shown in FIG. 8).

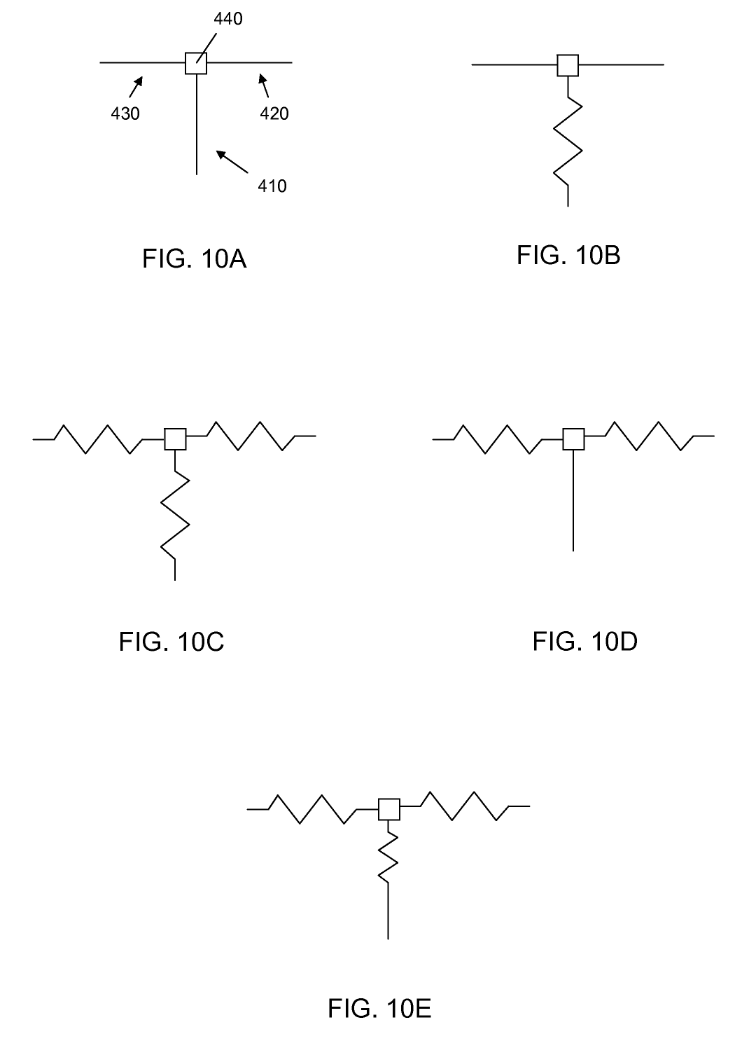

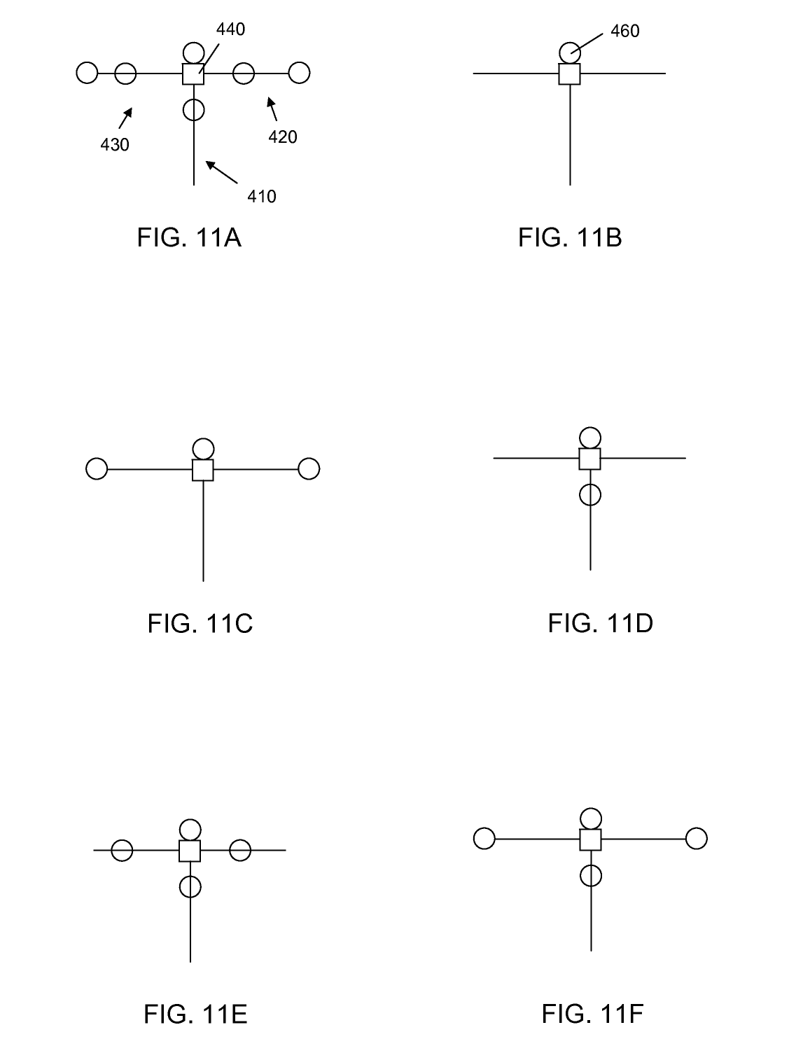

Referring now to FIGS. 10-11, various representative configurations of bifurcated leads are shown. While T-shaped configurations are depicted, it will be understood that such configurations are readily applicable to Y- or other shaped configurations. In the embodiments depicted in FIGS. 10A-E, the bifurcated leads include a proximal portion 410 containing contacts (not shown), a branch region 440 and first 420 and second 430 distal arms containing electrodes (not shown). The squiggly lines depicted in FIGS. 10B-E represent extensibility of the lead that the squiggly portion. Extensibility may include a sigma shaped section, loops, or may otherwise be configured to be extensible. As depicted, proximal portion 410 or distal arms 420, 430 or portions thereof may be extensible.

As shown in FIGS. 11A-F, in which circles represent anchors 460 that may be attached or unattached, a bifurcated lead may include one or more anchor at nearly any location of the lead, such as the distal portion or along the length of a distal arm 420, 430, at a branch region 440, or anywhere along the proximal portion 410. It will be understood that possible combinations of the configurations shown in FIGS. 10-11 are contemplated, as are combinations of other figured depicted and discussed herein.

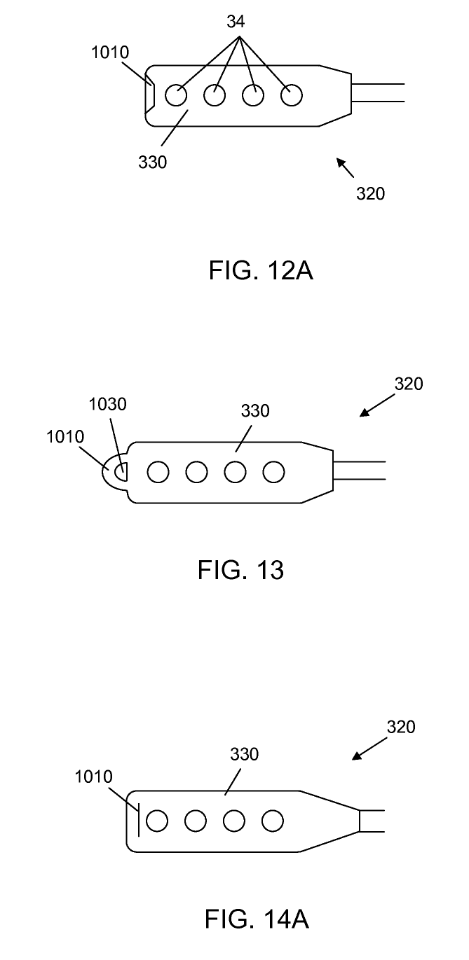

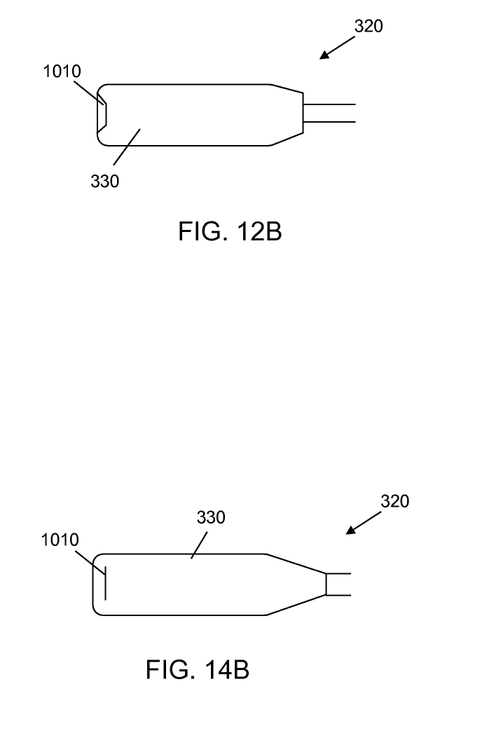

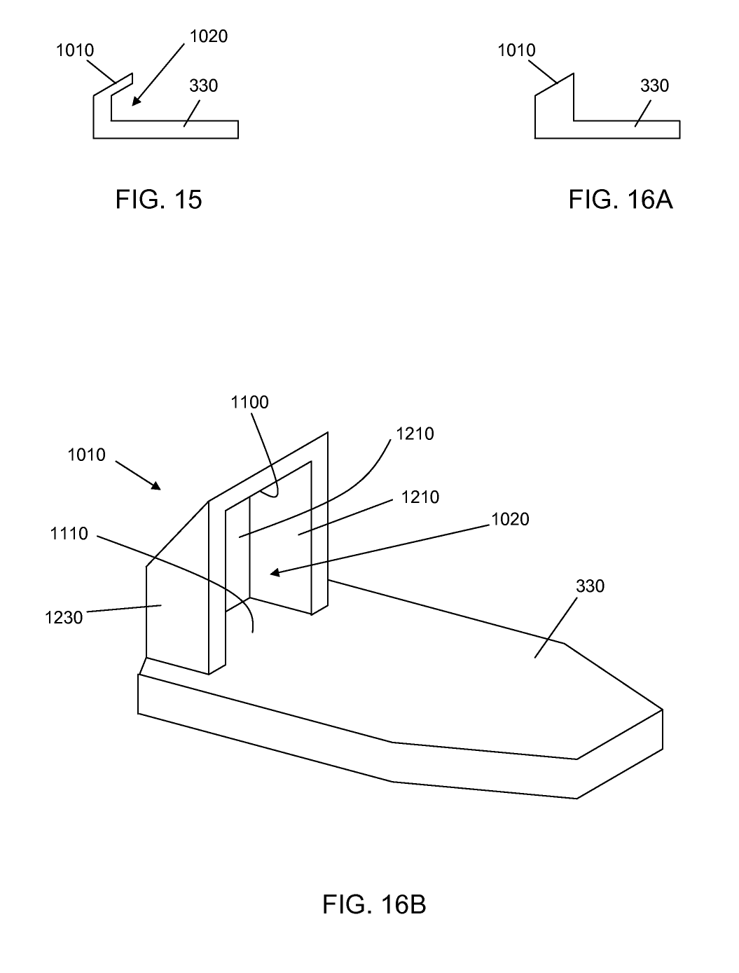

Referring now to FIGS. 12-16, various schematic views of distal portions of distal portions 320 (which correspond to distal arms 420, 430 in the figures described above) having engagement elements 1010 are shown. As shown in FIG. 12A, the distal portions 320 having one or more electrodes 34. As further shown in FIG. 12A, the depicted distal portions 320 may include paddle-shaped portions 330. The paddle shaped portion 330 includes the one or more electrodes 34 and the engagement element 1010. The engagement element 1010 is distal to the distal most electrode. The engagement element 1010 may be integrally formed with the paddle-shaped portion 330 or attached to the paddle-shaped portion (e.g., adhered, fastened, or otherwise secured).

With reference to FIGS. 12A, 13, and 14A, schematic top-down views of representative distal portions 320 of leads having a variety of engagement element 1010 configurations are shown. As depicted in FIG. 13, the engagement element 1010 may form a hole that may be engaged by a lead advancement tool tool, such as a tool having a hook. In the embodiment depicted in FIG. 14A, the engagement element 1010 includes or consists of a slit in the paddle-shaped portion 330 of the lead. A lead advancement tool may be inserted into the body of the paddle 330 to push the paddle to a desired implant location. In the embodiments depicted in FIGS. 12A and 14A, the engagement element 1010 extends from or is on the surface of the paddle 330 through which the electrodes are exposed. Typically paddle-shaped leads have electrodes exposed through one surface of the paddle, but not through the opposing surface. As shown in the embodiments depicted in FIGS. 12B and 14B, an engagement element 1010 may alternatively or additionally extends from, or may be on, the opposing surface of the paddle 330 through which the electrodes are not exposed.

Referring now to FIGS. 15 and 16A, schematic side views of alternative embodiments the distal portion of the lead depicted in FIG. 12B are show. The engagement element 1010 extends from a major surface of the paddle 330. As depicted in FIG. 15, the engagement element 1010 forms a cavity 1020 configured to receive an engagement tool.

Referring to FIG. 16B, a schematic perspective view of an embodiment of the paddle-shaped portion 330 of the lead depicted in FIG. 16A is shown. As with the engagement element depicted in FIG. 15, the engagement element 1010 depicted in FIG. 16A forms a cavity configured to receive an engagement tool. The cavity 1020 depicted in FIG. 16A is formed by first 1210, second 1220, and third 1230 side walls, a floor 1110, which may be even with the major surface of the paddle 330 or may be recessed relative to the major surface, and a ceiling 1100. The cavity 1020 depicted in FIG. 16B, or other similar cavities, allow the portion of an engagement tool received by the cavity 1020 to engage a variety of surfaces 1100, 1110, 1210, 1220, 1230 to allow for steering or guiding of the distal portion of the lead as it is pushed through tissue of a patient by the tool.

It will be understood that the engagement elements 1010 depicted in FIGS. 12-16 are merely examples engagement elements that may be employed in accordance with the teaching presented herein. Any other engagement element having a suitable configuration for engaging a portion of an engagement tool such that, when engaged by the tool, distal advancement of the tool pushes the distal portion of the lead distally.

It will be further understood that a lead engagement element may be positioned at any suitable location of the distal portion of the lead. Placing the engagement element distal to the distal most electrode or at or near the distal end of the lead allows for the remainder of the lead to be pulled through the patient's tissue by the pushing force applied to the distally located engagement element. However, if the lead is suitably designed (e.g., sufficiently rigid) to be pushed from a more proximal location, the engagement element may be place in a location more proximal than at or near the distal end of the lead. It will be further understood that the percutaneous leads, having generally cylindrical distal portions, or leads other that surgical or paddle leads may include engagement elements and may be implanted as described herein.

Engagement elements may be formed of any suitable material. In various embodiments, an engagement element is formed of material that forms the body of the paddle, such as polymeric material. Reinforcing elements may be included in the engagement members to provide sufficient structural rigidity to allow the lead to be pushed through tissue of the patient.

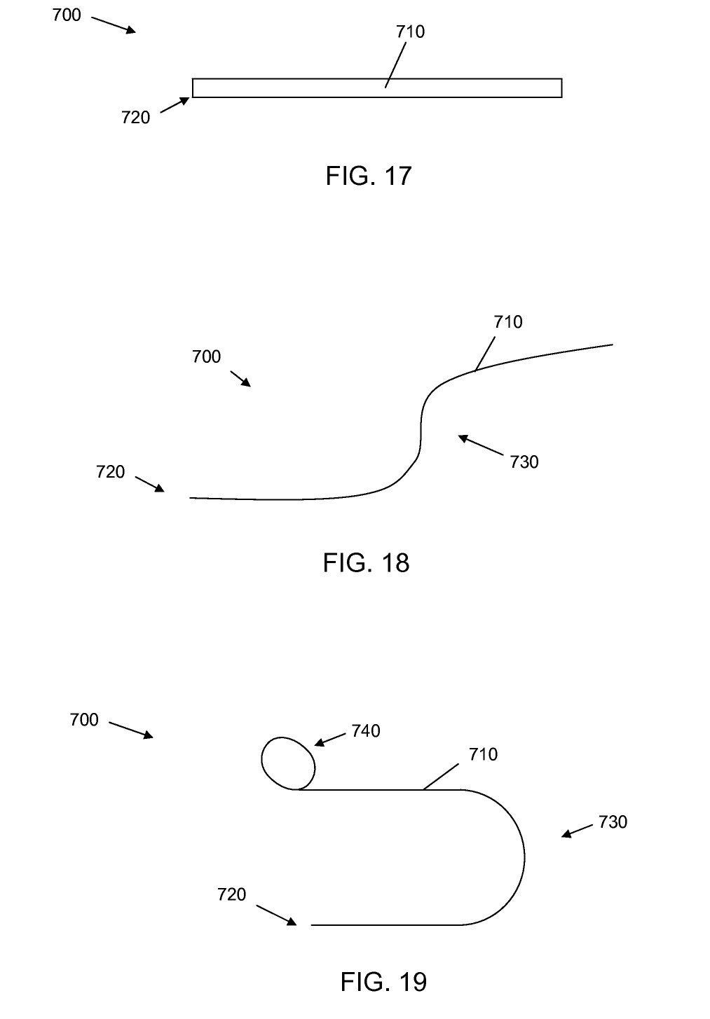

Referring now to FIGS. 17-19, schematic side views of alternative embodiments of engagement tools 700 are shown. The tools 700 have a lead engagement feature 720 configured to engage an engagement element of a lead. The tools 700 also include elongate members 710 that extend proximally from the lead engagement feature 720. In various embodiments, the lead engagement feature 720 is the distal end of the elongate member 710. As shown in FIGS. 18-19, the elongate members may include a curved portion 730. In some embodiments, the tools 700 are preformed to include the curved portion 730. In some embodiments, the elongate members 710 are configured to be manually bent to include a curve portion 730, as needed or desired, by a physician or other health care provider during the implant procedure. The tool 700 depicted in FIG. 19 is bent in a manner such that pulling on a portion, such as the loop 740, of the elongate member 710 distal to the engagement feature 720 cause a portion of the elongate member 710 proximal to the engagement feature 720 to push the engagement feature.

It will be understood that FIGS. 17-19 depict only some examples of suitable configurations for engagement tools that may be employed as described herein. Any other suitable form or configuration of engagement tool may be employed.

An engagement tool may be formed from any suitable material, such as a rigid polymeric material, a metallic material, combinations thereof, or the like. Preferably, the engagement tool is formed of material sufficiently stiff to push a lead through subcutaneous tissue of a patient, yet flexible enough to bend as may be needed during implantation.

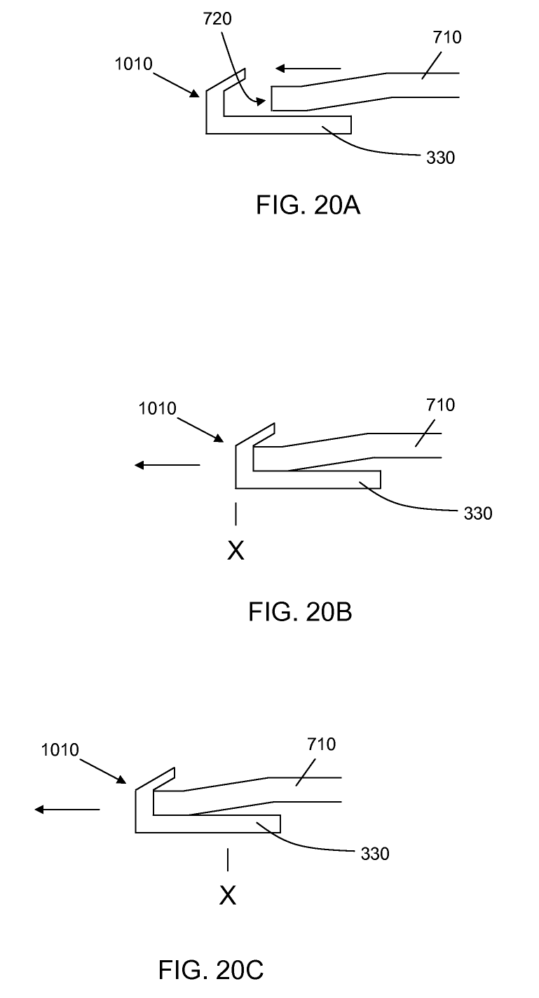

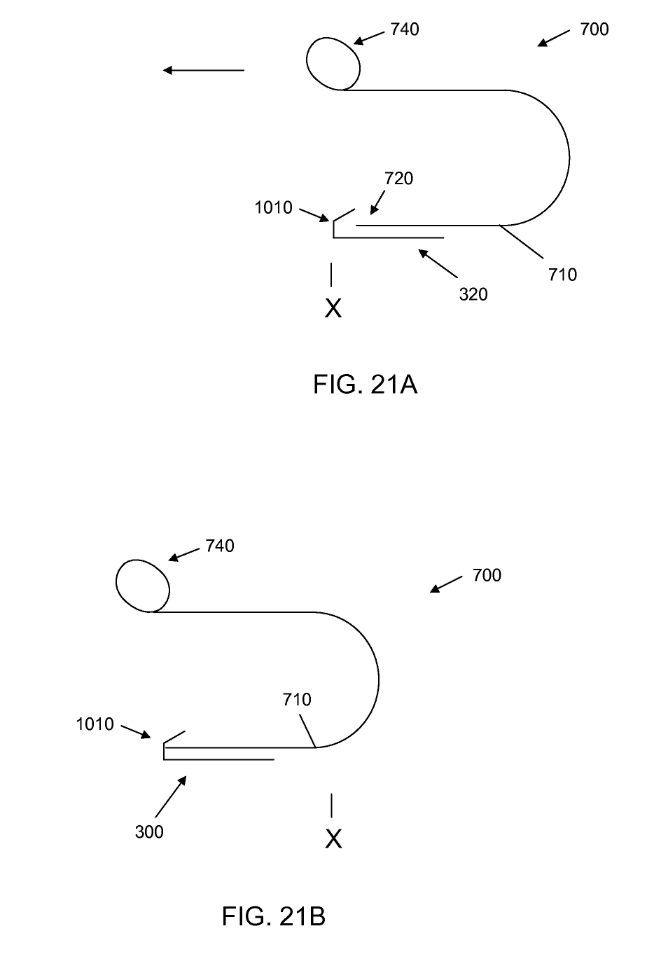

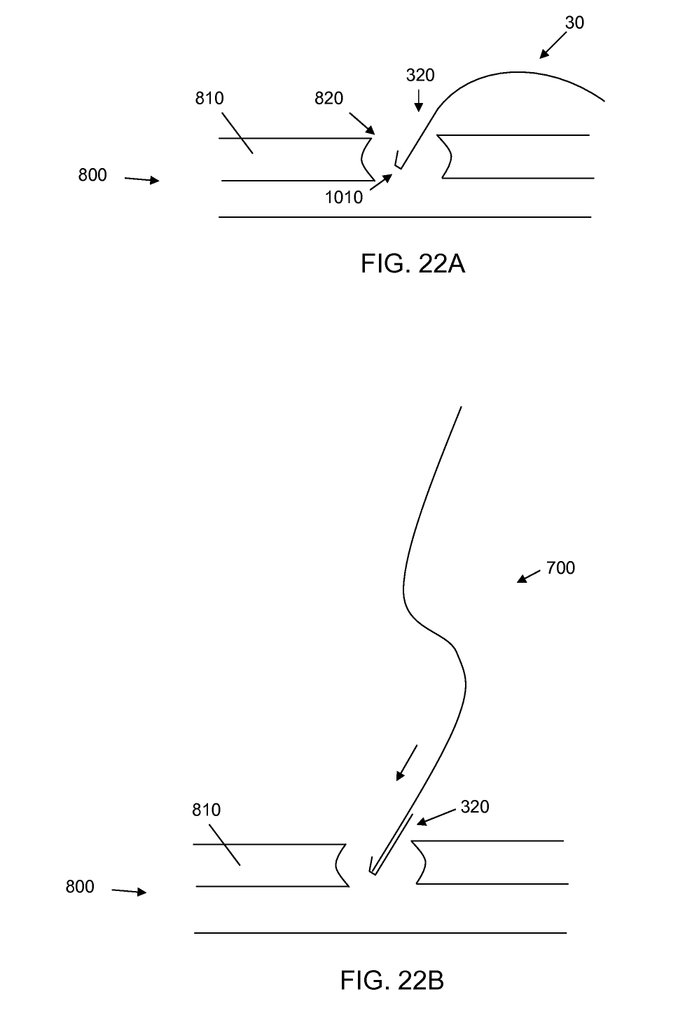

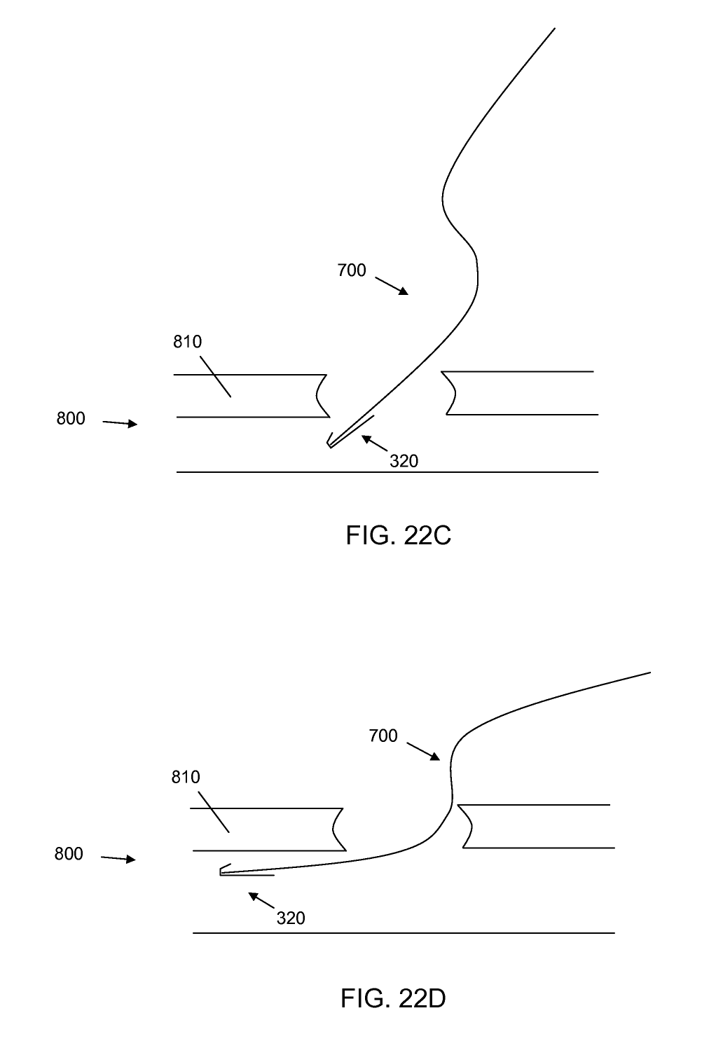

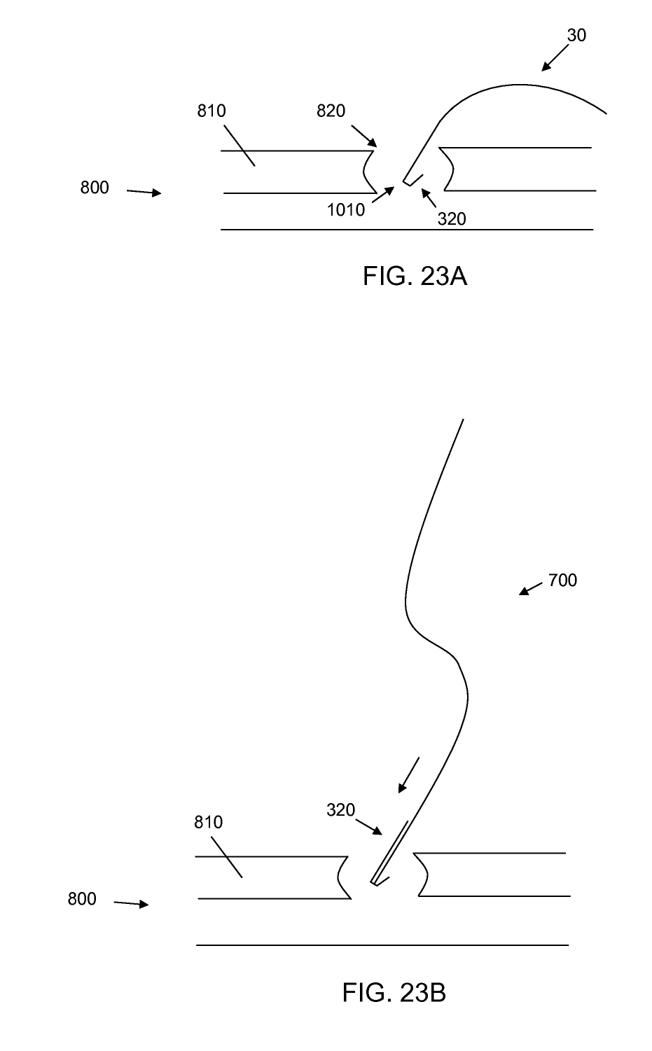

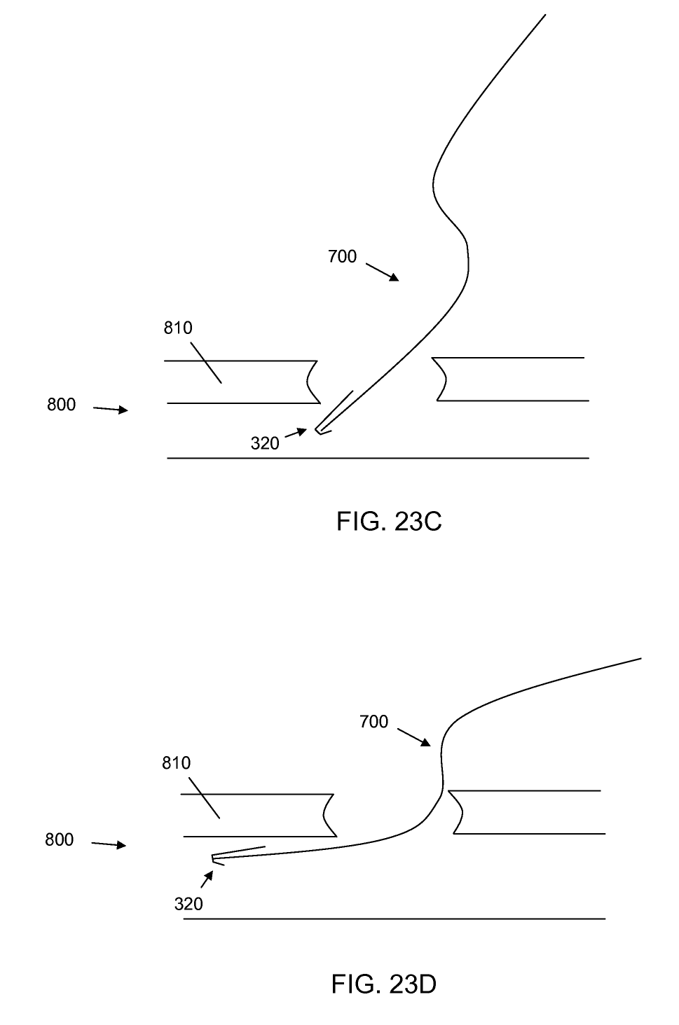

Referring now to FIGS. 20A-C, side views illustrating a tool pushing a distal portion of a lead (only distal portion shown for purposes of brevity, simplicity, and clarity). As shown in FIG. 20A-B, the elongate member 710 in proximity to the engagement feature 720 of a tool may be advanced distally relative to the lead until the engagement feature engages the engagement member 1010 of the paddle-shaped portion 330 of the lead. As shown in FIGS. 20B-C, further distal advancement of the elongate member 710 relative to the lead, when the tool is engaged with the engagement element 1010, causes the distal portion of the lead (including the paddle 330 in the depicted embodiment) to move distally. Position “X” indicated in FIGS. 20B-C is intended to mark a stationary position to reflect movement of the paddle portion 330 of the lead, and the elongate member 710 is pushed against the engagement feature 1010.

FIGS. 21A-B illustrate another example of a tool 700 moving a lead (only the distal portion 320 is shown for the purposes of brevity, simplicity, and clarity). The elongate member 710 distal to the engagement element 720 is pulled, e.g. by pulling on loop 740, to cause the elongate member 710 in proximity to the engagement feature 720 of the tool 700 to push the engagement feature 720. When the engagement feature 720 engages the engagement element 1010 at the distal portion 320 of the lead, distal advancement of the tool, causes the distal portion 320 of the lead to be moved distally.

Referring now to FIGS. 22A-D and FIGS. 23A-D, schematic drawings illustrating the advancement of a distal portion 320 of a lead 30 through tissue of a subject are shown. FIGS. 23A-D are substantially the same as FIGS. 22A-D, except that the orientation of the lead 30 is slightly different. It will be understood that only the distal portion 320 of the lead is shown in FIGS. 22B-D and FIGS. 23B-D for purposes of brevity, simplicity and clarity. As in FIGS. 20-21, the distal portion 320 of the lead includes and engagement element 1010 configured to cooperate with a tool 700 to advance the distal portion 320 of the lead through tissue 800 of a patient. The distal portion 320 of the lead 30 may be inserted through an incision 820 made in the patient. In the depicted embodiment, the incision 820 is through the skin 810 allowing advancement and implantation of the lead 30 in subcutaneous tissue 800 of the patient. A tool 700 (e.g. as described above) may be used to facilitate initial insertion into the subcutaneous tissue 800 (see, e.g., FIG. 22B, 23B) and is used to advance the distal portion 320 of the lead through the tissue 300 (see, e.g., FIGS. 22C-D, 23C-D). As the distal portion 320 of the lead enters the tissue 800 and is pushed through the tissue 800, the angle of the tool 700 (compare FIGS. 22B-D, 23B-D) is manipulated to implant the distal portion 320 of the lead at the appropriate angle and depth within the tissue 800. In the depicted embodiment, the tool 700 is pre-bent or curved. However, in various embodiments, the tool 700 may be bent or curved manually as needed or desired. Once the distal portion 320 of the lead is advanced to the desired location within the tissue 800, the tool 700 may be removed.

In some embodiments, the tool may be removed simply by withdrawing the tool from the tissue. However, in some embodiments, the engagement element of the lead and the engagement feature of the tool may be configured such that a significant amount of force is needed to disengage the tool from the engagement element of the lead (e.g., a compression fit, interference fit, snap fit, or the like). In such embodiments, it may be necessary to employ another tool to hold the distal portion on the lead in place while the engagement tool is disengaged to prevent movement of the distal portion of the lead from its desired implant location. Any suitable additional tool, such as forceps, pliers or the like to hold the paddle portion or the like, may be employed.

Thus, embodiments of BIFURCATED LEAD SYSTEM AND APPARATUS are disclosed. One skilled in the art will appreciate that the leads, extensions, connectors, devices such as signal generators, systems and methods described herein can be practiced with embodiments other than those disclosed. The disclosed embodiments are presented for purposes of illustration and not limitation.

1. A method for applying electrical signals to a left occipital nerve and a right occipital nerve of a subject, the method comprising:
providing a lead including (i) a proximal portion having first and second contacts and (ii) first and second distal arms,
wherein the first distal arm comprises a first electrode, and the second distal arm comprises a second electrode, and
wherein the first electrode is electrically coupled to the first contact and the second electrode is electrically coupled to the second contact;
placing the first electrode in electrical communication with the right occipital nerve;
placing the second electrode in electrical communication with the left occipital nerve;
generating a first electrical signal in an electrical signal generator implanted in the subject, wherein the electrical signal generator is operably coupled to the lead via the first contact;
applying the first electrical signal to the right occipital nerve via the first electrode;
generating a second electrical signal in an electrical signal generator implanted in the subject, wherein the electrical signal generator is operably coupled to the lead via the second contact;
applying the second electrical signal to the left occipital nerve via the second electrode of the lead,
wherein the first and second electrical signals are the same or different.

2. A method according to claim 1, wherein the lead includes (i) a branch region between the proximal portion and the first and second distal arms, and (ii) an tissue anchoring element attached to the branch region,
wherein the first distal arm of the lead includes an engagement element distal to the electrode, wherein the engagement element is configured to cooperate with a tool to facilitate placement of the lead such that distal advancement of the tool relative to the engagement feature pushes the first arm distally, and
wherein the method further comprises:
(i) anchoring the branch region to tissue of the patient; and
(ii) advancing the tool to push the first distal arm in the patient until the first electrode is in electrical communication with the right occipital nerve.

3. A method according to claim 2, wherein the second distal arm of the lead includes an engagement element distal to the electrode, wherein the engagement element is configured to cooperate with a tool to facilitate placement of the lead such that distal advancement of the tool relative to the engagement feature pushes the second arm distally, and
wherein the method further comprises advancing the tool to push the second distal arm in the patient until the second electrode is in electrical communication with the left occipital nerve.

4. A method according to claim 1, further comprising tunneling the proximal portion of the lead between a location of the subject nearer the left and right occipital nerves and a location at which the signal generator is implanted or is to be implanted.

5. A method according to claim 1, further comprising anchoring a portion of the lead in proximity to the branch region to tissue of the subject.

6. A bifurcated lead comprising:
a proximal portion having first and second contacts;
a first distal arm having a first electrode electrically coupled to the first contact and having a first engagement element distal the electrode, wherein the engagement element is configure to cooperate with an advancement tool such that advancement of the tool distally relative to the engagement element pushes the engagement element distally;
a second distal arm having a second electrode electrically coupled to the second contact and having a second engagement element distal the electrode, wherein the engagement element is configure to cooperate with an advancement tool such that advancement of the tool distally relative to the engagement element pushes the engagement element distally;
a branch region where the lead transitions from the proximal portion to the first and second distal arms; and
a tissue anchoring element attached to the branch region.

7. A lead according to claim 6, wherein the anchoring element is integrally formed with the branch region.

8. A lead according to claim 6, wherein the anchoring element comprises a suture loop.

9. A lead according to claim 6, wherein the first engagement element forms a cavity configured to receive a portion of the advancement tool.

10. A lead according to claim 6, wherein the second engagement element forms a cavity configured to receive a portion of the advancement tool.
